(19) United States
(12) Patent Application Publication
(10) Pub. No.: US 2023/0323271 A1
(43) Pub. Date: Oct. 12, 2023

(54) STERILE FOAM BREAKING SYSTEM AND ASSOCIATED METHOD THEREOF

(71) Applicant: GLOBAL LIFE SCIENCES SOLUTIONS USA LLC, Marlborough, MA (US)

(72) Inventors: Saravanan BALAKRISHNAN, Hoodi Village, Bengaluru (IN); Kandakumar MURUGESAN, Hoodi Village, Bengaluru (IN); Nagaraj Raghavendra RAO, Hoodi Village, Bengaluru (IN); Shashidhar HEGDE, Hoodi Village, Bengaluru (IN)

(21) Appl. No.: 18/044,273

(22) PCT Filed: Oct. 21, 2021
(86) PCT No.: PCT/EP2021/079286

§ 371 Date: Mar. 7, 2023

(30) Foreign Application Priority Data
Nov. 3, 2020 (IN) 202041047997

(51) Int. Cl. C12M 1/21; B01D 19/02

(52) U.S. Cl. CPC C12M 41/02; B01D 19/02

(57) ABSTRACT

A sterile foam breaking system, (14, 42, 52) includes a foam collector (20) having an opening (26), configured to be disposed in a source (12) which generates foam. The sterile foam breaking system (14, 42, 52) further includes a non-coated type suction unit (23) coupled to the foam collector (20). The non-contact type suction unit (23) is configured to transfer the foam via the opening (26) of the foam collector (20) and break a portion of the foam to generate a first quantity of liquid droplets. The sterile foam breaking system (14, 42, 52) additionally includes a foam breaking unit (28, 44, 54) coupled to the non-contact type suction unit (23). The foam breaking unit (28, 44, 54) is configured to receive remaining portion of the foam and the first quantity of liquid droplets and break the remaining portion of the foam to generate a second quantity of liquid droplets.

### FIELD OF INVENTION

The embodiments of the present specification relate generally to foam breaking systems, and more particularly, to a bioprocessing system having a foam breaking system and an associated method.

### BACKGROUND OF INVENTION

Liquid foams include gas bubbles which are closely packed within a liquid carrier matrix. A resulting network of gas/liquid interfaces provides liquid foams with numerous properties. Foams may also occur as a byproduct in many natural or industrial processes. In the latter case, their presence may interfere with production in a harmful manner; for example, in paper industry, paint industry, bioprocessing applications.

Specifically, with reference to bioprocessing applications, cell culture has generated considerable interest in recent years due to revolution in genetic engineering and biotechnology. Cells are cultured to make, for example, proteins, vaccines, and antibodies for therapy, research, and diagnostics. Specifically, a bioreactor is used to process biological materials (for example, to grow plant, animal cells, or the like) including, for example, mammalian, plant or insect cells and microbial cultures. Such devices may also be used for sterile mixing as well as non-sterile mixing applications. Some traditional bioreactors are designed as stationary pressurized vessels which can be mixed by several alternative means. Some other traditional bioreactors are designed as disposable bioreactors which utilize plastic sterile bags inside a culture vessel made from stainless steel or glass.

Cell culture in a bioreactor produces lot of foam due to reaction of Pluronic with sparged air. The generated foam fills head space of the bioreactor, thereby reducing an area for aerial oxygen mass transfer. Further, in some instances, foam can clog filter(s) of the bioprocessing system which would increase air pressure in the bioreactor, thereby affecting the bag and the entire cell culture medium. More often, cells and nutrients are carried away by the foam resulting in loss of expensive cultured products such as media, cells etc. Conventionally, anti-foam agent(s) are used for controlling the generation of foam. However, use of excess anti-foam agent(s) may reduce a mass transfer rate and a gas transfer coefficient, contaminate media and cause cell death, and affect separation and purification processes of the cells. Furthermore, the use of excess anti-foam agent(s) may cause malfunction of other downstream equipment/components.

There is a need for an enhanced system and method for overcoming the drawbacks discussed herein.

### BRIEF DESCRIPTION OF INVENTION

In accordance with one embodiment, a sterile foam breaking system includes a foam collector having an opening, configured to be disposed in a source which generates foam. The sterile foam breaking system further includes a non-contact type suction unit coupled to the foam collector. The non-contact type suction unit is configured to transfer the foam via the opening of the foam collector from the source and break a portion of the foam to generate a first quantity of liquid droplets. The sterile foam breaking system additionally includes a foam breaking unit coupled to the non-contact type suction unit. The foam breaking unit is configured to receive a remaining portion of the foam and the first quantity of liquid droplets and break the remaining portion of the foam to generate a second quantity of liquid droplets. The non-contact type suction unit is further configured to transfer the first quantity of liquid droplets and the second quantity of liquid droplets from the foam breaking unit to a collection vessel.

In accordance with another embodiment, a bioprocessing system having a bioreactor which generates foam and a sterile foam breaking system is disclosed.

In accordance with yet another embodiment, a method for operating a sterile foam breaking system is disclosed. The method includes transferring foam, by a non-contact type suction unit, via an opening of a foam collector from the source, wherein the foam collector is disposed in the source. The method further includes breaking, by the non-contact type suction unit, a portion of the foam to generate a first quantity of liquid droplets. The method also includes receiving, by a foam breaking unit, a remaining portion of the foam and the first quantity of liquid droplets from the non-contact type suction unit. The method additionally includes breaking, by the foam breaking unit, the remaining portion of the foam to generate a second quantity of liquid droplets. Additionally, the method includes transferring, by the non-contact type suction unit, the first quantity of liquid droplets and the second quantity of liquid droplets from the foam breaking unit to a collection vessel

### BRIEF DESCRIPTION OF DRAWINGS

These and other features, aspects, and advantages of the present disclosure will become better understood when the following detailed description is read with reference to the accompanying drawings in which like characters represent like parts throughout the drawings, wherein:

FIG. 1 is a schematic diagram of a system having a source, for example, a bioreactor and a foam breaking system having a serrated tube in accordance with an exemplary embodiment;

FIG. 2 is a partial perspective view of a foam breaking unit of the system in accordance with an embodiment of FIG. 1;

FIG. 3 is a schematic diagram of a system having a source, for example, a bioreactor and a foam breaking system having an agitator in accordance with an exemplary embodiment; and

FIG. 4 is a schematic diagram of a system having a source, for example, a bioreactor and a foam breaking system having a pressure chamber in accordance with an exemplary embodiment; and

FIG. 5 is a flow chart illustrating a method for operating a system having a source and a foam breaking system in accordance with an exemplary embodiment.

### DETAILED DESCRIPTION

For the purpose of promoting an understanding of the principles of the disclosure, reference will now be made to embodiments illustrated in the figures and specific language will be used to describe them. It will nevertheless be understood that no limitation of the scope of the disclosure is thereby intended. Such alterations and further modifications to the disclosure, and such further applications of the principles of the disclosure as described herein being contemplated as would normally occur to one skilled in the art to which the disclosure relates are deemed to be a part of this disclosure.

It will be understood by those skilled in the art that the foregoing general description and the following detailed description are exemplary and explanatory of the disclosure and are not intended to be restrictive thereof.

Unless defined otherwise, technical and scientific terms used herein have the same meaning as is commonly understood by one of ordinary skill in the art to which this disclosure belongs. The terms “first,” “second,” and the like, as used herein do not denote any order, quantity, or importance, but rather are used to distinguish one element from another. The terms “comprises”, “comprising”, or any other variations thereof, are intended to cover a non-exclusive inclusion, such that a process or method that comprises a list of steps does not include only those steps but may include other steps not expressly listed or inherent to such a process or a method. Similarly, one or more devices or sub-systems or elements or structures or components preceded by “comprises . . . a” does not, without more constraints, preclude the existence of other devices, other sub-systems, other elements, other structures, other components, additional devices, additional sub-systems, additional elements, additional structures, or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this disclosure belongs. The system, methods, and examples provided herein are illustrative only and not intended to be limiting.

Embodiments of the present disclosure will be described below in detail with reference to the accompanying figures.

In accordance with one embodiment of the present disclosure, a sterile foam breaking system is disclosed. The sterile foam breaking system includes a foam collector having an opening. The foam collector is configured to be disposed in a source which generates foam. The sterile foam breaking system further includes a non-contact type suction unit coupled to the foam collector. The non-contact type suction unit is configured to transfer the foam via the opening of the foam collector from the source and break a portion of the foam to generate a first quantity of liquid droplets. The sterile foam breaking system further includes a foam breaking unit coupled to the non-contact type suction unit. The foam breaking unit is configured to receive a remaining portion of the foam and the first quantity of liquid droplets from the non-contact type suction unit and break the remaining portion of the foam to generate a second quantity of liquid droplets. The non-contact type suction unit is further configured to transfer the first quantity of liquid droplets and the second quantity of liquid droplets from the foam breaking unit to the source (or another collection vessel). In one embodiment, the foam breaking system may be used in a bioprocessing system having a bioreactor. In certain other embodiments, the foam breaking system may be used for any other sources which generates foam. In another embodiment, a method for operating a foam breaking system is disclosed.

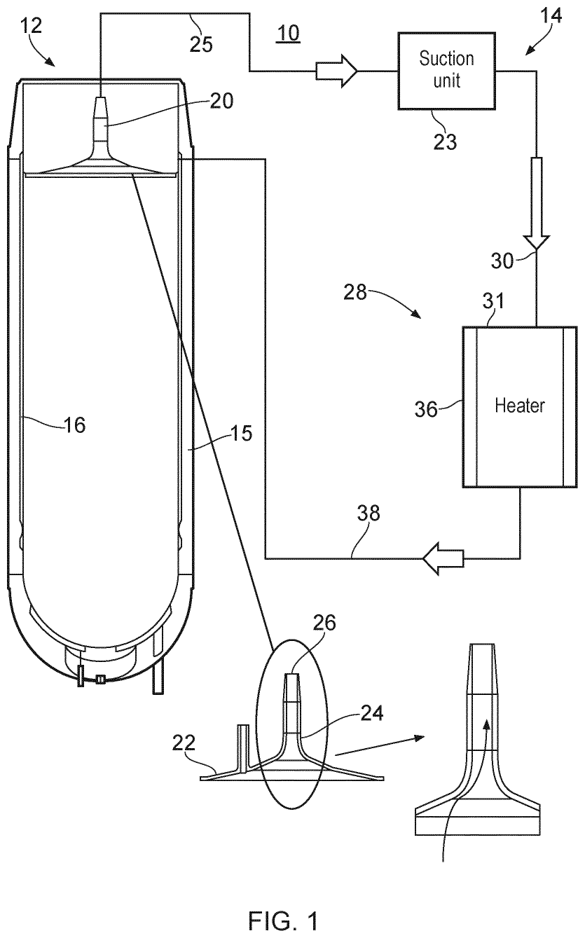

FIG. 1 is schematic block diagram of a system 10 having a source 12, for example, a bioreactor and a foam breaking system 14 in accordance with an exemplary embodiment. In the illustrated embodiment, the system 10 is a bioprocessing system. In other embodiments, the system 10 may be other types of system used for other applications such as in a chemical industry, for example, where foam is generated.

In the illustrated embodiment, the source 12 (i.e. bioreactor) includes a base module (not shown) and the vessel 15 configured to support and substantially enclose an enclosure 16. In one embodiment, the enclosure 16 is a bag, for example, a bioreactor bag. The size of the vessel 15 may vary depending on the application. In another embodiment, the enclosure 16 may a container, for example, a metal container such as a stainless-steel container. In such an embodiment, the vessel 15 may not be required.

The base module includes a base support (not shown) and an impeller drive unit (not shown) disposed within the base support. The vessel 15 includes a mating connection device (not shown) coupled to a corresponding mating connection device (not shown) of the base support. Hence, the vessel 15 is stably supported by the base support. The vessel 15 may include a cylindrical side wall having a plurality of side walls coupled to each other via a plurality of hinges. One of the second side walls can be opened to access interior of the vessel 15 and for loading and unloading the enclosure 16. The diameter of the cylindrical side wall may vary depending on the application. In another embodiment, the vessel 15 may have a single integrated cylindrical side wall instead of a plurality of side walls.

In one embodiment, the plurality of side walls may be manufactured by plastic injection molding. In one specific embodiment, thermoplastic material can be used for molding the side walls of the vessel 15. In another embodiment, the side walls of the vessel 15 may be formed by stamping sheet metal or by 3D printing of either plastic or metal.

Furthermore, with reference to the plurality of hinges, opposite side edges of the side walls are provided with a locking device (not shown) so that the side walls can be detachably locked in a closed position. In one embodiment, the locking device may include co-operating magnets provided on side edges of the second side walls. In another embodiment, the locking device may include a snap lock or external standard latches to lock the first and second side walls against each other in a closed position. In another embodiment, the vessel 15 may have a side wall of different configuration, for example, a square shaped side wall instead of a cylindrical side wall. It should be noted herein that the vessel 15 discussed herein in an exemplary embodiment and should not be construed as a limitation of the scope of the disclosure. Other suitable designs of the vessel are also envisioned within the scope of the disclosure.

The vessel 15 may include one or more flexible heater pads (not shown) provided on an inner surface of the cylindrical side wall. The flexible heater pads are configured to heat the enclosure 16 when the enclosure 16 is loaded within the vessel 15. In some embodiments, the flexible heater pads are provided symmetrically around the loaded enclosure 16. In one embodiment, the flexible heater pads are made of but not limited to silicone, polyimide, or other flexible heat-resistant polymers disposed enclosing electrical heating elements which typically are conductive fibers or films. In some embodiments, the vessel 15 can additionally include a flexible cooling jacket provided to the cylindrical side wall.

Further, the enclosure 16 is provided with a mixing unit (not shown). Furthermore, in one embodiment, the enclosure 16 is filled with a fluid medium such as but not limited to a culture medium. The mixing unit is configured to perform mixing of the fluid medium within the enclosure 16.

In the illustrated embodiment, the sterile foam breaking system 14 is coupled to the source 12. The sterile foam breaking system 14 includes a foam collector 20 disposed in the enclosure 16. In one embodiment, the foam collector 20 is a floating type foam collector which floats on the fluid medium filled in the enclosure 16 such that in use, the foam collector 20 is located at a liquid gas interface within the enclosure 16. The foam collector 20 includes a flat portion 22 and a protruding portion 24 extending upwards from the flat portion 22. The flat portion 22 is configured for contacting the fluid medium. The protruding portion 24 has a through-opening 26 for permitting flow of generated foam from the fluid medium. According to certain embodiments, the foam collector 20 has a frustoconical shape. In other words, the flat portion 22 is generally circular in shape and open in the interior and tapers up to the protruding portion 24, as illustrated. Specifically, the inside of the foam collector 20 has a funnel-type shape, a larger end starting at the flat portion 22 and ending at the through-opening 26. The number of protruding portions 24 may also vary depending on the application. It should also be noted herein that the configuration and shape of the foam collector 20 may also vary depending on the application. In one embodiment, the foam collector 20 may be made of a plastic material, such as suitable biocompatible polymers, for example, polyethylene. In other embodiments, the foam collector 20 may be made of any other suitable rigid or semi-rigid biocompatible material that is capable of floating at a liquid gas interface and being sterilized. Further, in certain other embodiments, the foam collector 20 may be made of more than one material. For example, the flat portion 22 may be made of a biocompatible material, such as a suitable polymer, while the at least a portion of the protruding portion 24 may be made from a metallic material (since the protruding portion 24 does not directly contact with the fluid medium).

The sterile foam breaking system 14 further includes a non-contact type suction unit 23 coupled to the foam collector 20 via a sterile first connection path 25. In the illustrated embodiment, the non-contact type suction unit 23 is a non-contact type suction pump. The non-contact type suction unit 23 is configured to transfer the foam via the through-opening 26 of the foam collector 20 from the source 12 and break a portion of the foam to generate a first quantity of liquid droplets. Specifically, the non-contact type suction unit 23 sucks the generated foam via the through-opening 26 in the foam collector 20 due to differential pressure generated by the non-contact type suction unit 23. A portion of the foam breaks due to pumping pressure of the non-contact type suction unit 23. In certain other embodiments, other types of non-contact type suction units are also envisioned. Further, the sterile foam breaking system 14 includes a foam breaking unit 28 coupled to the non-contact type suction unit 23 via a second connection path 30. The foam breaking unit 28 is configured to receive a remaining portion of the foam and the first quantity of liquid droplets from the non-contact type suction unit 23 and break the remaining portion of the foam to generate a second quantity of liquid droplets. In the illustrated embodiment, the foam breaking unit 28 includes a tube 31, one or more meshes (not shown in FIG. 1) disposed within the tube 31, and a plurality of serrations (not shown in FIG. 1) formed within the tube 31. The remaining portion of the foam is broken to generate a second quantity of liquid droplets by foam breaking unit 28. Furthermore, the foam breaking unit 28 includes an optional heater 36 coupled to an external surface of the tube 31 to heat the tube 31. The warm surface of the tube 31 facilitates to increase kinetic energy of air particles in the remaining portion of the foam locally, thereby reducing surface tension and hence break the remaining portion of the foam to generate a second quantity of liquid droplets. The foam breaking unit 28 is also coupled to the source 12 via the third connection path 38. The non-contact type suction unit 23 is further configured to transfer the first quantity of liquid droplets and the second quantity of liquid droplets from the foam breaking unit 28 to the source 12, thereby conserving the expensive media and cells in the fluid medium. In the illustrated embodiment, the source 12 is a collection vessel. According to further embodiments, at least one check valve (not shown) or other flow control device can be provided in at least one of the connection paths 25, 30, 38 to ensure that the foam and liquid droplets can only flow along a single direction as illustrated by the arrows. For example, one-way valves may be placed in or otherwise incorporated in at least one of the connection paths 25, 30, 38. Additionally, the third connection path 38 can be coupled to another collection vessel (not shown) instead of the source 12. In such an embodiment, such a collection vessel is different from the source 12.

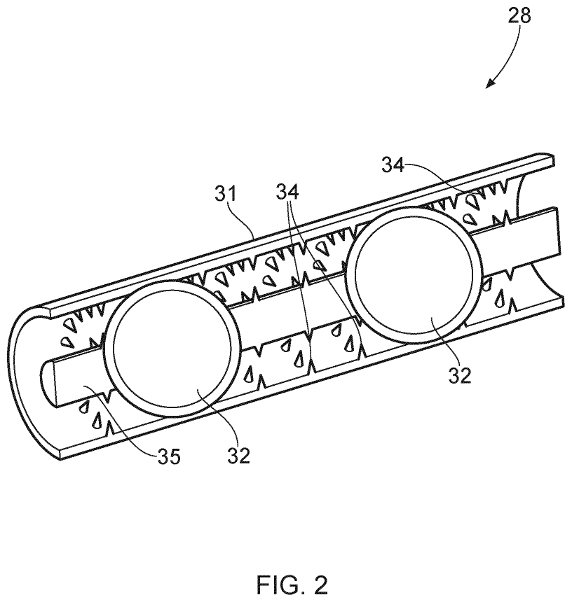

FIG. 2 is a partial perspective view of the foam breaking unit 28 of the system 10 in accordance with an embodiment of FIG. 1. In the illustrated embodiment, the foam breaking unit 28 includes the tube 31, one or more meshes 32 disposed within the tube 31, and a plurality of serrations 34 formed within the tube 31. In an embodiment, the tube 31 may be made of stainless steel. In another embodiment, the tube 31 may be made of plastic. The material of the tube 31 may vary depending on the application. In one embodiment, only the one or more meshes 32 are used instead of serrations 34 within the tube 31. The number of meshes 32 and spacing between the meshes 32 may vary depending on the application. In another embodiment, only the serrations 34 may be used instead of the one or more meshes 32 within the tube 31. The number of serrations 34 and spacing between the serrations 34 may vary depending on the application. The remaining portion of the foam is broken to generate a second quantity of liquid droplets due to frictional effect of the one or more meshes 32 and the serrations 34. As also illustrated by FIG. 2, the tube 31 may include a central support structure 35 that extends along the length of the tube 31 through its approximate center and includes at least one of the serrations 34. In another embodiment, the tube 31 may not include the central support structure 35.

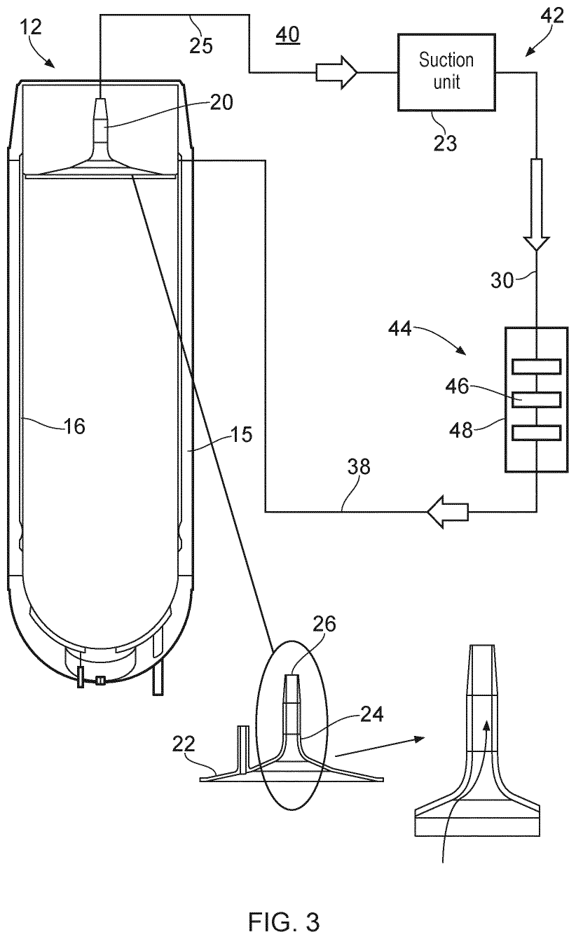

FIG. 3 is schematic block diagram of a system 40 having the source 12, for example, a bioreactor and a foam breaking system 42 in accordance with another exemplary embodiment. In the illustrated embodiment, the system 40 is a bioprocessing system. In other embodiments, the system 40 may be other types of system used for other applications such as in a chemical industry, for example, where foam is generated. In the illustrated embodiment, the source 12 (i.e. bioreactor) includes a base module (not shown) and the vessel 15 configured to support and substantially enclose an enclosure 16.

In the illustrated embodiment, the sterile foam breaking system 42 is coupled to the source 12. The sterile foam breaking system 42 includes the foam collector 20 disposed in the enclosure 16. In one embodiment, the foam collector 20 is a floating type foam collector which floats on the fluid medium filled in the enclosure 16. The foam collector 20 includes the flat portion 22 and the protruding portion 24 extending upwards from the flat portion 22. The flat portion 22 is disposed contacting the fluid medium. The protruding portion 24 has the through-opening 26 for permitting flow of generated foam from the fluid medium.

The sterile foam breaking system 42 further includes the non-contact type suction unit 23 coupled to the foam collector 20 via the sterile first connection path 25. The non-contact type suction unit 23 is configured to transfer the foam via the through-opening 26 of the foam collector 20 from the source 12 and break the portion of the foam to generate the first quantity of liquid droplets. Specifically, the non-contact type suction unit 23 sucks the generated foam via the through-opening 26 in the foam collector 20 due to differential pressure generated by the non-contact type suction unit 23. A portion of the foam breaks due to pumping pressure of the non-contact type suction unit 23. Further, the sterile foam breaking system 42 includes a foam breaking unit 44 coupled to the non-contact type suction unit 23 via the second connection path 30. The foam breaking unit 44 is configured to receive a remaining portion of the foam and the first quantity of liquid droplets from the non-contact type suction unit 23 and break the remaining portion of the foam to generate the second quantity of liquid droplets. In the illustrated embodiment, the foam breaking unit 44 is an agitator. The foam breaking unit 44 includes at least one impeller 46 disposed within a casing 48. The remaining portion of the foam is broken to generate the second quantity of liquid droplets due to agitation by the impeller 46. The agitator may be but not limited to a mechanical agitator, static agitator, rotating tank agitator, magnetic agitator, or the like. Furthermore, the foam breaking unit 44 may include an optional heater 36 (shown in FIG. 1) coupled to an external surface of the casing 48 to heat the casing 48. The warm surface of the casing 48 facilitates to increase kinetic energy of air particles in the remaining portion of the foam locally, thereby reducing surface tension and hence break the remaining portion of the foam to generate a second quantity of liquid droplets. The foam breaking unit 44 is also coupled to the source 12 via the third connection path 38. The non-contact type suction unit 23 is further configured to transfer the first quantity of liquid droplets and the second quantity of liquid droplets from the foam breaking unit 44 to the source 12. In another embodiment, the non-contact type suction unit 23 is further configured to transfer the first quantity of liquid droplets and the second quantity of liquid droplets from the foam breaking unit 44 to another collection vessel instead of the source 12.

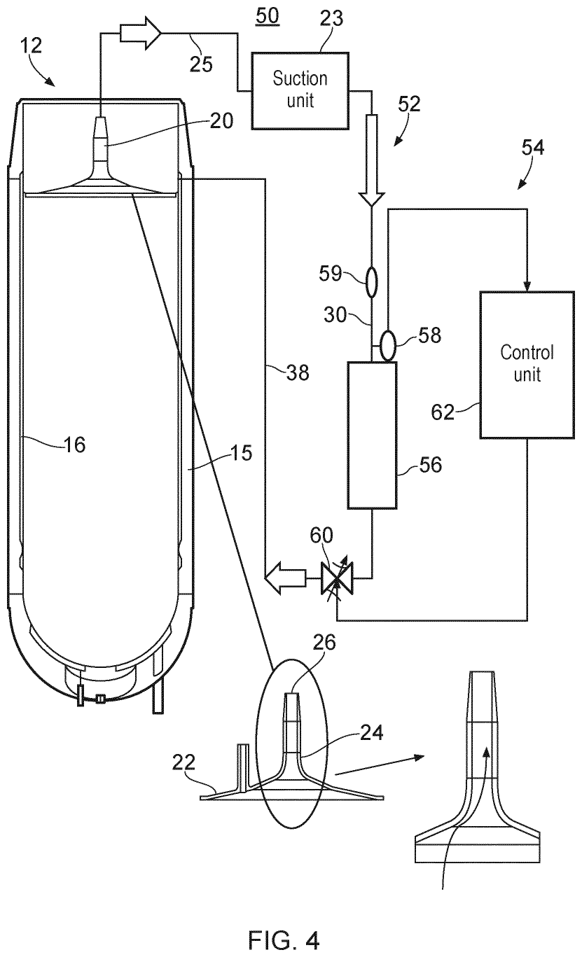

FIG. 4 is a schematic block diagram of a system 50 having the source 12, for example, a bioreactor and a foam breaking system 52 in accordance with yet another exemplary embodiment. In the illustrated embodiment, the system 50 is a bioprocessing system. In other embodiments, the system 50 may be other types of system used for other applications such as in a chemical industry, for example, where foam is generated.

In the illustrated embodiment, the sterile foam breaking system 52 is coupled to the source 12. The sterile foam breaking system 52 includes the foam collector 20 disposed in the enclosure 16. The foam collector 20 includes the flat portion 22 and the protruding portion 24 extending upwards from the flat portion 22. The flat portion 22 is disposed contacting the fluid medium. The protruding portion 24 has the through-opening 26 for permitting flow of generated foam from the fluid medium.

The sterile foam breaking system 52 further includes the non-contact type suction unit 23 coupled to the foam collector 20 via the sterile first connection path 25. The non-contact type suction unit 23 is configured to transfer the foam via the through-opening 26 of the foam collector 20 from the source 12 and break the portion of the foam to generate the first quantity of liquid droplets. Specifically, the non-contact type suction unit 23 sucks the generated foam via the through-opening 26 in the foam collector 20 due to differential pressure generated by the non-contact type suction unit 23. A portion of the foam breaks due to pumping pressure of the non-contact type suction unit 23. Further, the sterile foam breaking system 52 includes a foam breaking unit 54 coupled to the non-contact type suction unit 23 via the second connection path 30. The foam breaking unit 54 is configured to receive a remaining portion of the foam and the first quantity of liquid droplets from the non-contact type suction unit 23 and break the remaining portion of the foam to generate the second quantity of liquid droplets. The foam breaking unit 54 is also coupled to the source 12 via the third connection path 38.

In the illustrated embodiment, the foam breaking unit 54 includes a pressure chamber 56, a pressure sensor 58 disposed upstream of the pressure chamber 56, a one-directional flow control valve 59 disposed upstream of the pressure chamber 56, and a proportional pinch flow control valve 60 disposed downstream of the pressure chamber 56.

The pressure sensor 58 is configured to detect a pressure in the pressure chamber 56. Specifically, the one-directional flow control valve 59 is coupled to the second connection path 30. The one-directional flow control valve 59 is configured to control a flow of the remaining portion of the foam and the first quantity of liquid droplets from the non-contact type suction unit 23 to the pressure chamber 56. The remaining portion of the foam is broken to generate the second quantity of liquid droplets due to relatively higher pressure within the pressure chamber 56. Also, specifically, the proportional pinch flow control valve 60 is coupled to the third connection path 38. The proportional pinch flow control valve 60 is configured to control a flow of the first quantity of liquid droplets and the second quantity of liquid droplets from the pressure chamber 56 to the source 12.

Furthermore, in some embodiments, the foam breaking unit 54 may include an optional heater 36 (shown in FIG. 1) coupled to an external surface of the pressure chamber 56 to heat the pressure chamber 56. The warm surface of the pressure chamber 56 facilitates to increase kinetic energy of air particles in the remaining portion of the foam locally, thereby reducing surface tension and hence break the remaining portion of the foam to generate a second quantity of liquid droplets. The non-contact type suction unit 23 is further configured to transfer the first quantity of liquid droplets and the second quantity of liquid droplets from the foam breaking unit 54 to the source 12. In another embodiment, the non-contact type suction unit 23 is further configured to transfer the first quantity of liquid droplets and the second quantity of liquid droplets from the foam breaking unit 54 to another collection vessel instead of the source 12

Additionally, the foam breaking unit 54 includes a control unit 62 communicatively coupled to the pressure sensor 58 and the proportional pinch flow control valve 60. The control unit 62 is configured to control the proportional pinch flow control valve 60 based on an output of the pressure sensor 58. In one embodiment, when a detected pressure within the pressure chamber 56 exceeds a threshold limit, the control unit 62 opens the proportional pinch flow control valve 60 to transfer the first quantity of liquid droplets and the second quantity of liquid droplets from the foam breaking unit 54 to the source 12. When the detected pressure within the pressure chamber 56 is less than a threshold limit, the control unit 62 closes the proportional pinch flow control valve 60. In one embodiment, the one-directional flow control valve 59 may also be controlled by the control unit 62.

In one embodiment, the control unit 62 includes at least one of a general-purpose computer, a graphics processing unit (GPU), a digital signal processor, and a controller. In some embodiments, the control unit 62 may be implemented as one or more microprocessors, microcomputers, microcontrollers, digital signal processors, central processing units, state machines, logic circuitries, and/or any device that manipulates signals based on operational instructions. Among other capabilities, the at least one processor is configured to fetch and execute computer-readable instructions stored in the memory unit. In other embodiments, the control unit 62 includes a customized processor element such as, but not limited to, an application-specific integrated circuit (ASIC) and a field-programmable gate array (FPGA). In some embodiments, the control unit 62 may be communicatively coupled with at least one of a keyboard, a mouse, and any other input device and configured to receive commands and/or parameters from an operator via a console.

In one embodiment, the memory unit is a random-access memory (RAM), a read only memory (ROM), a flash memory, or any other type of computer readable memory accessible by the processor. In some embodiments, the memory unit may include, for example, volatile memory such as static random access memory (SRAM) and/or dynamic random access memory (DRAM) and/or non-volatile memory such as read only memory (ROM), erasable programmable ROM, flash memories, hard disks, optical disks, and/or magnetic tapes. Also, in certain embodiments, the memory unit may be a non-transitory computer readable medium encoded with a program having a plurality of instructions to instruct the processor to operate one or more valves.

In certain embodiments, the control unit 62 may include an I/O interface having a variety of client application and hardware interfaces, for example, a web interface, a graphical user interface, and the like. The I/O interface may allow the control unit 62 to interact with a customer directly or through customer devices. Further, the I/O interface may enable the control unit 62 to communicate with other computing devices such as web servers and external data servers (not shown). The I/O interface may facilitate multiple communications within a wide variety of networks and protocol types, including wired networks such as Local Area Network, cable, etc., and wireless networks such as Wireless Local Area Network, cellular, satellite, etc. The I/O interface may include one or more ports for connecting a plurality of devices to each other and/or to another server.

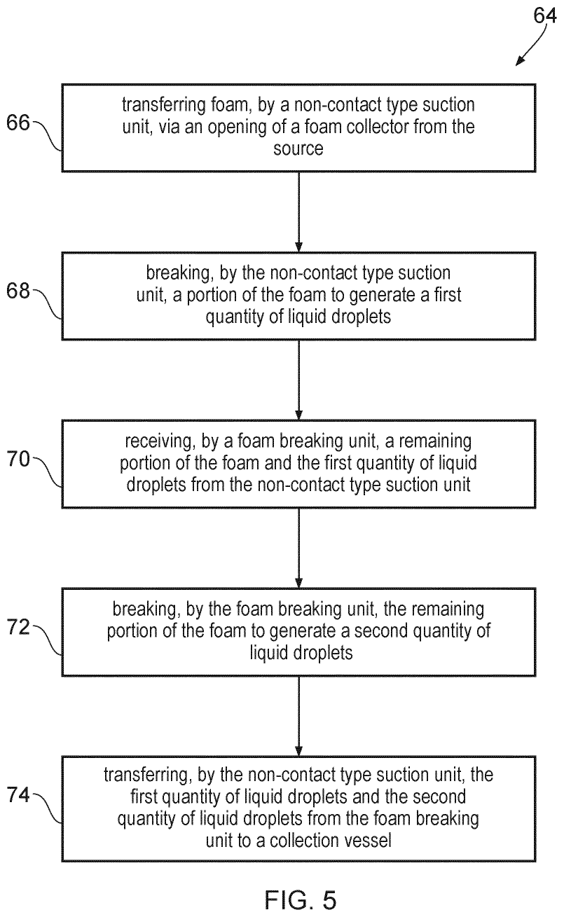

FIG. 5 is a flow chart illustrating a method 64 for operating a system having a source and a foam breaking system in accordance with an exemplary embodiment. The non-contact type suction unit transfers the generated foam via the through-opening of the foam collector from the source as represented by the step 66. The non-contact type suction unit breaks a portion of the foam to generate a first quantity of liquid droplets as represented step 68. Specifically, the non-contact type suction unit sucks the generated foam via the through-opening in the foam collector due to differential pressure generated by the non-contact type suction unit. A portion of the foam breaks due to pumping pressure of the non-contact type suction unit. The foam breaking unit receives a remaining portion of the foam and the first quantity of liquid droplets from the non-contact type suction unit as represented by step 70. The foam breaking unit breaks the remaining portion of the foam to generate a second quantity of liquid droplets. The remaining portion of the foam is broken to generate a second quantity of liquid droplets by foam breaking unit as represented by the step 72. In one embodiment, the remaining portion of the foam is broken by a mesh, a plurality of serrations, or a combination thereof, within a tube to generate the second quantity of liquid droplets. In another embodiment, the remaining portion of the foam is broken by an agitator to generate the second quantity of liquid droplets. In yet another embodiment, the remaining portion of the foam is broken within a pressure chamber to generate the second quantity of liquid droplets.

Optionally, a heater may be used to enhance the breaking the remaining portion of the foam to generate the second quantity of liquid droplets within the foam breaking unit. The non-contact type suction unit is further used to transfer the first quantity of liquid droplets and the second quantity of liquid droplets from the foam breaking unit to the source as represented by the step 74. In another embodiment, the non-contact type suction unit is further configured to transfer the first quantity of liquid droplets and the second quantity of liquid droplets from the foam breaking unit to another collection vessel instead of the source 12

In accordance with the embodiments discussed herein, an exemplary foam breaking system is used to remove generated foam from a source, break the generated foam to liquid droplets, and then transfer the liquid droplets back to the source. The exemplary foam breaking system is configured to overcome drawbacks associated with aerial oxygen mass transfer and clogging of filter(s). Further, the exemplary system and process facilitates to minimize adverse effects on the bag and the entire cell culture medium. Also, there is minimal losses of expensive cultured products such as media, cells, etc. Further, there is no need to use an anti-foaming agent, thereby eliminating drawbacks associated with use of anti-foaming agent.

While only certain features of the specification have been illustrated and described herein, many modifications and changes will occur to those skilled in the art. It is, therefore, to be understood that the appended claims are intended to cover all such modifications and changes as fall within the true spirit of the specification.

1. A sterile foam breaking system comprising:
a foam collector comprising an opening, wherein the foam collector is configured to be disposed in a source which generates foam;
a non-contact type suction unit coupled to the foam collector, wherein the non-contact type suction unit is configured to:
transfer the foam via the opening of the foam collector from the source; and
break a portion of the foam to generate a first quantity of liquid droplets; and
a foam breaking unit coupled to the non-contact type suction unit wherein the foam breaking unit is configured to:
receive a remaining portion of the foam and the first quantity of liquid droplets from the non-contact type suction unit; and
break the remaining portion of the foam to generate a second quantity of liquid droplets;
wherein the non-contact type suction unit is further configured to transfer the first quantity of liquid droplets and the second quantity of liquid droplets from the foam breaking unit to a collection vessel.

2. The sterile foam breaking system as claimed in claim 1, wherein the foam collector is a floating type foam collector comprising a flat portion and a protruding portion comprising the opening, extending from the flat portion.

3. The sterile foam breaking system as claimed in claim 1, wherein the foam collector is made of plastic.

4. The sterile foam breaking system as claimed in claim 1, wherein the foam breaking unit comprises:
a tube; and
a mesh disposed within the tube a plurality of serrations formed within the tube, or a combination thereof.

5. The sterile foam breaking system as claimed in claim 4, wherein the foam breaking unit further comprises a heater coupled to an external surface of the tube.

6. The sterile foam breaking system as claimed in claim 4, wherein the tube is made of stainless steel.

7. The sterile foam breaking system as claimed in claim 4, wherein the tube is made of plastic.

8. The sterile foam breaking system as claimed in claim 1, wherein the foam breaking unit comprises an agitator.

9. The sterile foam breaking system as claimed in claim 1, wherein the foam breaking unit further comprises a heater coupled to an external surface of the agitator.

10. The sterile foam breaking system as claimed in claim 1, wherein the foam breaking unit comprises:
a pressure chamber;
a one-directional flow control valve disposed upstream of the pressure chamber, wherein the one-directional flow control valve is configured to control a flow of the remaining portion of the foam and the first quantity of liquid droplets from the non-contact type suction unit to the pressure chamber;
a pressure sensor disposed upstream of the pressure chamber, wherein the pressure sensor is configured to detect a pressure in the pressure chamber;
a proportional pinch flow control valve disposed downstream of the pressure chamber, wherein the proportional pinch flow control valve, is configured to control a flow of the first quantity of liquid droplets and the second quantity of liquid droplets from the pressure chamber to the collection vessel; and
a control unit communicatively coupled to the pressure sensor and the proportional pinch flow control valve wherein the control unit is configured to control the proportional pinch flow control valve based on an output of the pressure sensor.

11. The sterile foam breaking system as claimed in claim 10, wherein the foam breaking unit further comprises a heater coupled to an external surface of the pressure chamber.

12. A bioprocessing system comprising:
a bioreactor which generates foam; and
a sterile foam breaking system coupled to the bioreactor; wherein the sterile foam breaking system comprises:
a foam collector comprising an opening, wherein the foam collector is disposed in the bioreactor;
a non-contact type suction unit coupled to the foam collector, wherein the non-contact type suction unit is configured to:
transfer the foam via the opening of the foam collector from the bioreactor; and
break a portion of the foam to generate a first quantity of liquid droplets; and
a foam breaking unit coupled to the non-contact type suction unit, wherein the foam breaking unit is configured to:
receive a remaining portion of the foam and the first quantity of liquid droplets from the non-contact type suction unit; and
break the remaining portion of the foam to generate a second quantity of liquid droplets;
wherein the non-contact type suction unit is further configured to transfer the first quantity of liquid droplets and the second quantity of liquid droplets from the foam breaking unit to the bioreactor.

13. The bioprocessing system as claimed in claim 12, wherein the foam collector is a floating type foam collector comprising a flat portion and a protruding portion comprising the opening, extending from the flat portion wherein the flat portion contacts a fluid medium filled in the bioreactor.

14. A method for operating a sterile foam breaking system, the method comprising:
transferring foam, by a non-contact type suction unit, via an opening of a foam collector from the source, wherein the foam collector cy is disposed in the source;
breaking, by the non-contact type suction unit, a portion of the foam to generate a first quantity of liquid droplets;
receiving, by a foam breaking unit, a remaining portion of the foam and the first quantity of liquid droplets from the non-contact type suction unit;
breaking, by the foam breaking unit, the remaining portion of the foam to generate a second quantity of liquid droplets; and
transferring, by the non-contact type suction unit, the first quantity of liquid droplets and the second quantity of liquid droplets from the foam breaking unit to a collection vessel.

15. The method as claimed in claim 14, wherein breaking, by the foam breaking unit, the remaining portion of the foam to generate the second quantity of liquid droplets comprises breaking, by a mesh, a plurality of serrations, or a combination thereof, the remaining portion of the foam within a tube to generate the second quantity of liquid droplets.

16. The method as claimed in claim 14, wherein breaking, by the foam breaking unit, the remaining portion of the foam to generate the second quantity of liquid droplets comprises breaking, by an agitator, the remaining portion of the foam to generate the second quantity of liquid droplets.

17. The method as claimed in claim 14, wherein breaking, by the foam breaking unit, the remaining portion of the foam to generate the second quantity of liquid droplets comprises breaking, within a pressure chamber, the remaining portion of the foam to generate the second quantity of liquid droplets.

18. The method as claimed in claim 17, further comprising:
controlling, by a one-directional flow control valve, a flow of the remaining portion of the foam and the first quantity of liquid droplets from the non-contact type suction unit to the pressure chamber;
detecting, by a pressure sensor, a pressure in the pressure chamber; and
controlling, by a control unit, a proportional pinch flow control valve based on an output of the pressure sensor to control a flow of the first quantity of liquid droplets and the second quantity of liquid droplets from the pressure chamber to the collection vessel.
